(19) United States
(12) Patent Application Publication
(10) Pub. No.: US 2024/0237961 A1
(43) Pub. Date: Jul. 18, 2024

(54) A Surface Audio-Visual Biofeedback (SAVB) System for Motion Management

(71) Applicants: Tomi NANO, San Francisco, CA (US); Dante CAPALDI, San Francisco, CA (US); The Regents of the University of California, Oakland, CA (US); The Board of Trustees of the Leland Stanford Junior University, Stanford, CA (US)

(72) Inventors: Tomi F. Nano, San Francisco, CA (US); Dante P.I. Capaldi, San Francisco, CA (US)

(21) Appl. No.: 18/579,775

(22) PCT Filed: Jul. 22, 2022
(86) PCT No.: PCT/US2022/074050

§ 371 Date: Jan. 16, 2024

Related U.S. Application Data
(60) Provisional application No. 63/225,171, filed on Jul. 23, 2021.

(51) Int. Cl. A61B 6/00; A61B 6/46; A61B 6/50; A61N 5/10

(52) U.S. Cl. CPC A61B 6/527; A61B 6/463; A61B 6/541; A61B 6/563; A61N 5/1049; A61B 6/502; A61N 2005/1059

(57) ABSTRACT

Methods, systems, and devices, including computer programs encoded on a computer storage medium are provided for measuring and displaying subject motion information during procedures which require remote subject monitoring. The system uses a mobile device with depth sensor capabilities, data processing capabilities and artificial intelligence (AI) predictive models to provide motion information. The system motion information can be used to measure the period of time a subject performed deep-inspiration breath hold (DIBH) and for training the subject to achieve a DIBH of at least 20 seconds.

### CROSS-REFERENCE TO RELATED APPLICATIONS

This application claims priority to United States Provisional Application Ser. No. 63/225,171 filed Jul. 23, 2021, the disclosure of which is herein incorporated by reference in its entirety.

### INTRODUCTION

According to the American Cancer Society, breast cancer is the second most common cancer for women and the second leading cause of cancer related deaths. Radiation therapy is highly successful at treating breast cancer by delivering a high therapeutic dose of radiation to the breast while limiting exposure to the healthy lungs and heart. Unfortunately, some women can experience adverse radiation effects and reduced survival if cardiac and lung doses are not maintained below a certain threshold; for every 1 Gy of radiation exposure to the heart, the relative risk of cardiac events increases by 7%. This can be challenging for women with left-sided breast cancer because the heart is directly adjacent and in close proximity to the breast under treatment. A strategy to reduce heart and lung dose, particularly for left-sided breast cancer patients, is to deliver radiation to the breast while the patient performs multiple deep-inspiration breath holds (DIBH), each of approximately 20 seconds or more duration. During DIBH the diaphragm descends and moves the heart further away from the chest wall receiving radiation; simultaneously, DIBH expands the lungs and reduces the amount of normal lung that is irradiated.

### SUMMARY

Methods, systems, and devices, including computer programs encoded on a computer storage medium, are provided for measuring and displaying motion and monitoring metrics related to the motion that are computed from motion sensor data. In certain aspects, artificial intelligence (AI) is used for measuring and/or displaying the motion and monitoring metrics. The methods, systems, and devices disclosed herein may be used for performing deep-inspiration breath hold (DIBH) radiation treatments on patients. In certain aspects, the methods, systems, and devices disclosed herein are used for measuring and displaying the period of time a subject can hold breath. Thus, these methods, systems, and devices find use in training a subject to achieve a clinically acceptable DIBH time.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1. The user interface of the iSAVB application for measuring motion traces and providing feedback. The ISAVB system incorporates the TrueDepth video data that has a depth color-map (left) and the corresponding respiratory trace from the averaged depth data (right). The depth data [cm] is a 1D signal which is the average pixel values in the center region of interest (ROI) (shown in the color map). The local view provides breath-hold guidance to the patient, while the remote view provides feedback to the treatment control area during treatment. The cloud system synchronizes these GUIs and sends the appropriate information to each view.

FIG. 2A and FIG. 2B. Comparison of the iSAVB system and the QUASAR™ motion phantom with various waveform settings. Periodic motion programmed on the motion phantom and measured using the iSAVB application shows excellent agreement for both free-breathing (FIG. 2A) and breath-hold traces (FIG. 2B). Red-line=IRF iOS application; black-line=RPM™ system. Relationship between iSAVB application and QUASAR motion phantom traces acquired were significantly correlated (free-breathing: r=0.999, r2=0.998, p<0.0001, slope=0.979; breath-hold: r=0.991, r2=0.982, p<0.0001, slope=0.941). Bland-Altman analysis of agreement for iSAVB and QUASAR motion phantom (free-breathing: bias=0.00±0.04 cm, lower limit=−0.07 cm, upper limit=0.07 cm; breath-hold: bias=0.00±0.08 cm, lower limit=−0.16 cm, upper limit=0.16 cm). Dotted lines indicate the 95% confidence interval.

FIG. 3. is a flow diagram illustrating steps for installation check performed by a system used for a method according to one embodiment of the present disclosure.

FIG. 4 is a flow diagram illustrating hardware compatibility check performed by a system used for a method according to one embodiment of the present disclosure.

FIG. 5 illustrates depth sensor functioning self-test.

FIG. 6 illustrates remote server (cloud) self-test.

FIG. 7 illustrates flowchart for depth map and motion graph display.

FIG. 8 illustrates motion modeling using artificial intelligence.

### DETAILED DESCRIPTION OF EMBODIMENTS

Methods, systems, and devices, including computer programs encoded on a computer storage medium, are provided for measuring and displaying motion and monitoring metrics related to the motion that are computed from motion sensor data. In certain aspects, artificial intelligence (AI) is used for measuring and/or displaying the motion and monitoring metrics. The methods, systems, and devices disclosed herein may be used for performing deep-inspiration breath hold (DIBH) radiation treatments on patients. In certain aspects, the methods, systems, and devices disclosed herein are used for measuring and displaying the period of time a subject can hold breath. Thus, these methods, systems, and devices find use in training a subject to achieve a clinically acceptable DIBH time.

Before the present methods, systems, and devices are described, it is to be understood that this invention is not limited to particular methods or compositions described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a period of time” includes a plurality of such periods of time and reference to “the period of time” includes reference to one or more periods of time and equivalents thereof, e.g., a first period of time, a second period of time, and so forth, which periods of time may be same of different in length.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The terms “determining”, “measuring”, “evaluating”, “assessing,” “assaying,” and “analyzing” are used interchangeably herein to refer to any form of measurement, and include determining if an element is present or not.

The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or symptom(s) from occurring in a subject who may be predisposed to the disease or symptom but has not yet been diagnosed as having it; (b) inhibiting the disease and/or symptom(s), i.e., arresting their development; or (c) relieving the disease symptom(s), i.e., causing regression of the disease and/or symptom(s). Those in need of treatment include those already inflicted (e.g., those with cancer, etc.) as well as those in which prevention is desired (e.g., those with increased susceptibility to cancer, those suspected of having cancer, those with a risk of recurrence, etc.).

A therapeutic treatment is one in which the subject has a condition/disease prior to administration and a prophylactic treatment is one in which the subject does not have a condition/disease prior to administration.

The terms “subject,” “individual” or “patient” are used interchangeably herein and refer to a human subject and include males and females who are adults or children.

### Methods

Methods, systems, and devices, including computer programs encoded on a computer storage medium, are provided for measuring and displaying motion and monitoring metrics related to the motion that are computed from motion sensor data. In certain aspects, artificial intelligence (AI) is used for measuring and/or displaying the motion and monitoring metrics. The methods, systems, and devices disclosed herein may be used for performing deep-inspiration breath hold (DIBH) radiation treatments on patients. In certain aspects, the methods, systems, and devices disclosed herein are used for measuring and displaying the period of time a subject can hold breath. Thus, these methods, systems, and devices find use in training a subject to achieve a clinically acceptable DIBH time.

A computer-implemented method for capturing motion of chest and/or abdomen associated with inhalation and exhalation of a subject by using a relatively easily available motion sensor, such aa, a motion/position capture device is disclosed. The method may include focusing a lens of a motion/position capture device on a region of interest (ROI) on the torso of a subject; capturing motion of the ROI over a period of time; and simultaneously generating a real-time motion trace comprising a plot of movement over time, wherein the plot captures motion of chest and/or abdomen of the subject associated with inhalation and exhalation.

In certain embodiments, the motion/position capture device may be a smartphone, e.g., a smartphone that operates on an Apple™ operating system or a smartphone that operates on other operating systems with depth sensor capabilities. The Apple™ operating system, iOS, may be iOS14.1 or iOS14.6, which includes a front-facing depth camera, or a back-facing light detection and ranging (LiDAR) sensor used for depth measurement.

In certain embodiments, the device may include a front-facing camera located on the same side of the device as the screen of the device or a back-facing camera located on the back side of the device that is on the other side of the screen. The plot may be displayed on the screen.

In certain embodiments, the subject may have a tumor located in the torso, neck or head. In certain embodiments, the tumor may be breast tumor.

The device may be connected to a data storage system, a remote display device, a radiation device, and/or a medical imaging device.

In certain embodiments, the data storage system may be cloud storage, the remote display device may be a computer monitor, laptop, smartphone, or another handheld device comprising a screen, and the medical imaging device may perform a computer-assisted tomography (CAT) scan, a magnetic resonance imaging, or positron emission tomography (PET) scan.

In certain embodiments, the method may include prompting the subject to perform deep-inspiration breath hold (DIBH) prior to start of the capturing and/or after the start of the capturing. In certain aspects, the capturing is performed during the entire duration of training the subject to perform DIBH.

In certain embodiments, the method may further include indicating visually or audibly to the subject a first period of time the subject performed DIBH based on the analysis of the real-time motion trace.

In certain embodiments, the method may include further prompting the subject to perform DIBH and indicating visually or audibly to the subject a second period of time the subject performed DIBH.

In certain embodiments, the steps of prompting may be repeated till the subject performs DIBH for a period of at least 20 seconds.

In certain embodiments, the method may further include indicating visually or audibly to a healthcare provider the periods of time the subject performed DIBH based on the analysis of the real-time motion trace.

The healthcare provider may be present at a location remote to the subject. The method may include uploading the real-time motion trace and/or the periods of time the subject performed DIBH to a data storage, wherein the data storage is accessible by the healthcare provider.

In certain embodiments, the method may include relaying exhalation after end of DIBH by the subject to a radiation device and/or an imaging device.

In certain embodiments, the method may include instructing a radiation device to pause radiation being delivered to the region of interest when the subject exhales after the end of DIBH.

In certain embodiments, the method may include instructing an imaging device to pause imaging of the region of interest when the subject exhales after the end of DIBH.

Also provided is a non-transitory computer-readable medium comprising instructions stored thereon for causing a computer system to implement the methods of the present disclosure.

A computer system comprising the non-transitory computer-readable medium is also disclosed.

The method can be implemented in digital electronic circuitry, or in computer software, firmware, or hardware. The disclosed and other embodiments can be implemented as one or more computer program products, i.e., one or more modules of computer program instructions encoded on a computer readable medium for execution by, or to control the operation of, a data processing apparatus, such as, a smartphone. The computer readable medium can be a machine-readable storage device, a machine-readable storage substrate, a memory device, a composition of matter effecting a machine-readable propagated signal, or any combination thereof. For example, a smartphone may include computer program instructions that when executed by the processor causes the smartphone to perform the methods disclosed herein.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

In a further aspect, a system for performing the computer implemented method, as described, is provided. Such a system includes a computer containing a processor, a storage component (i.e., memory), a display component, and other components typically present in general purpose computers. The storage component stores information accessible by the processor, including instructions that may be executed by the processor and data that may be retrieved, manipulated or stored by the processor.

The storage component includes instructions. The computer processor is coupled to the storage component and configured to execute the instructions stored in the storage component and analyze the data according to one or more algorithms (e.g., deep convolutional neural network or deep residual neural network). The display component displays information regarding the time period of DIBH in the individual.

The storage component may be of any type capable of storing information accessible by the processor, such as a hard-drive, memory card, ROM, RAM, DVD, CD-ROM, USB Flash drive, write-capable, and read-only memories. The processor may be any well-known processor, such as processors from Intel Corporation. Alternatively, the processor may be a dedicated controller such as an ASIC.

The instructions may be any set of instructions to be executed directly (such as machine code) or indirectly (such as scripts) by the processor. In that regard, the terms “instructions,” “steps” and “programs” may be used interchangeably herein. The instructions may be stored in object code form for direct processing by the processor, or in any other computer language including scripts or collections of independent source code modules that are interpreted on demand or compiled in advance.

Data may be retrieved, stored or modified by the processor in accordance with the instructions. For instance, although the system is not limited by any particular data structure, the data may be stored in computer registers, in a relational database as a table having a plurality of different fields and records, XML documents, or flat files. The data may also be formatted in any computer-readable format such as, but not limited to, binary values, ASCII or Unicode. Moreover, the data may comprise any information sufficient to identify the relevant information, such as numbers, descriptive text, proprietary codes, pointers, references to data stored in other memories (including other network locations) or information which is used by a function to calculate the relevant data.

In certain embodiments, the processor and storage component may comprise multiple processors and storage components that may or may not be stored within the same physical housing. For example, some of the instructions and data may be stored on removable CD-ROM and others within a read-only computer chip. Some or all of the instructions and data may be stored in a location physically remote from, yet still accessible by, the processor. Similarly, the processor may comprise a collection of processors which may or may not operate in parallel.

In certain embodiments, the plot for DIBH or the time periods for DIBH and/or other data content are shown to the subject via a display component, such as a television, a monitor, a high-definition television (HDTV), or a head-up display (HUD).

The motion capture device includes a depth camera. A non-limiting example of a motion capture device includes a video camera, such as an RGB video camera and/or a depth camera, such as, a camera present in iPhone12. Other mobile devices may have similar depth cameras. Alternatively, external device plugins that can be easily integrated into a mobile device. The motion/position capture device includes a depth camera that can sense distance between an imaging sensor in the camera and objects in the camera's field of view, in order to acquire a depth image of the subject. Depth images, color images, or both may be captured. If both color and depth images are captured, the color and depth images may be acquired simultaneously by a camera with two lenses, one for acquiring color images and one for acquiring depth images. A color image is a digital representation of an image which contains multiple channels, each channel corresponding to a different color. In certain aspects, three channels are used, and each channel corresponds to one of the colors red, green, and blue. However, any other suitable number of colors and color selection may be assigned to the multiple channels. Each channel is composed of an identical number of pixels, and each pixel has an intensity value between zero and a maximum number. The maximum number may vary depending upon the application of the images. The value of each pixel corresponds to the contribution of that color channel at each pixel's location.

A depth image may contain a single channel composed of the same number of pixels as each color channel. The value of each pixel in a depth image corresponds to the distance between the camera lens and the user at each corresponding pixel's location. Different approaches may be employed for generating depth images, including time of flight, stereoscopic vision, and triangulation. The color images and the depth images may be analyzed and processed independently.

The region of interest on a person's torso may be located on the surface of the chest over a region where a tumor is located. Three-dimensional coordinates for each one of the feature points of interest may be computed from color and/or depth images. The coordinate locations for each of the feature points of interest may be stored for the frame corresponding to co-acquired color and depth images.

### Utility

The methods described herein are useful for reducing radiation exposure of tissue adjacent to a tumor, e.g., lung and/or heart for a subject receiving radiation therapy for treatment of a tumor with deep-inspiration breath-hold (DIBH).

The methods described herein are also useful for improving medical imaging of a subject by, e.g., CAT scan, MRI, or PET scan of the torso region by reducing artifacts introduced by motion.

The methods, systems, and devices described herein are different from other remote motion monitoring systems because it uses a mobile platform with depth sensor capability and an algorithm that provides motion feedback. The method includes motion predictions from personalized AI models which will enable additional feedback for patients and clinician that improve overall treatment.

### Algorithm for Providing Motion Feedback

The algorithm was developed for mobile phones with LiDAR capability. More specifically, the LiDAR camera provides depth information, in addition to the scene information (i.e., image), that is used to obtain surface information of the object of interest. Both the depth and scene information are acquired as a function of time to create a video feed of both depth and scene to enable motion tracking of the object and displayed to the user. Region of interest (ROI) selection can be performed by the user in the mobile phone application graphical user interface (GUI). This ROI selection is projected on the video feeds to enable the user to properly select the area on the patient's body that is of interest. Once selected, the depth information is acquired specifically in this region and displayed as a one-dimensional motion trace over time by averaging the depth information in the ROI. Additionally, both the one-dimensional and two-dimensional depth information in the ROI over time is recorded and saved in an array. The two-dimensional depth information in the ROI is used to calculate the normal vector to a plane generated by three points in the selected ROI depth image to display in addition to the one-dimensional motion trace. Moreover, the two-dimensional depth information in the ROI is used to perform non-rigid surface registration on a reference surface (at the time of acquisition) to provide six-degree of motion (x, y, z, yaw, pitch, roll) information.

### Motion Predictions Based on Personalized AI Models

A long short-term memory (LSTM) artificial neural network was implemented to predicted motion for free-breathing one-dimensional motion trace. Specifically, once the user selects a specific ROI to track motion over, initialization of the LS™ model was performed to create the initial vector (xᵢ) used as an input to predict the next depth data point in the future (yᵢ). The input (xᵢ₊₁) is continuously updated in a sliding windowing manner in order to predict successive depth data points in the future. This iterative process enables a patient specific motion trace prediction model.

Personalized artificial intelligence model is advantageous as they provide patient specific information to both the health care providers as well as the patient for tailoring optimal treatment plans. Patient specific information in the form of predicting patient specific respiratory motion for radiation oncology purposes enables ideal delivery of radiation in anatomical regions susceptible to motion (e.g., thorax and abdomen). Specifically, these motion models can be used to turn off and on the radiation at specific parts of a patient's respiratory cycle to minimize the dose to surrounding normal tissue.

The method described herein can be independently used by patients, e.g., at home, to practice breathing maneuvers that will improve their clinical outcome from a treatment requiring DIBH. The training of the subject to successfully perform breathing maneuvers, such as DIBH, for a period of at least 20 seconds, decreases movement of the chest and/or abdominal wall of the subject during radiation therapy or imaging. In addition, the end of DIBH can be relayed to the radiation device or the medical imaging device, promoting the device to pause radiation or imaging, respectively. Clinically acceptable DIBH may be a time period of at least 3 seconds, at least 5 seconds, at least 8 seconds, at least 10 seconds, at least 13 seconds, at least 15 seconds, at least 18 seconds, at least 20 seconds, at least 25 seconds, e.g., between 10 seconds-30 seconds.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

1. A computer-implemented method comprising:
displaying on a screen of a motion/position capture device and/or on a screen remotely connected to the device:

(A) real-time depth video stream of the surface of the torso of a subject in a field of view of a camera of the motion/position capture device, and

(B) real-time plot of motion of the surface;
focusing the field of view to capture motion over a region of interest (ROI) on the surface;
capturing motion in the ROI over a period of time; and
displaying (A) the real-time depth video stream of the ROI and an adjusted (B) real-time plot of motion for the ROI, wherein the (B) real-time plot of motion for the ROI displays motion of chest and/or abdomen of the subject associated with inhalation and exhalation.

2. The method of aspect 1, wherein capturing motion in the ROI over a period of time comprises simultaneously updating (B) real-time plot of motion for the ROI by applying an artificial intelligence (AI) to update motion metrics.

3. The method of aspect 1 or 2, comprising simultaneously transmitting data comprising (A) the real-time depth video stream of the ROI and the adjusted (B) real-time plot of motion for the ROI to a remote server.

4. The method of any one of aspects 1-3, wherein the screen remotely connected to the device is the screen of a remote monitoring device, a data storage device, a radiation treatment device, and/or an imaging device.

5. The method of any one of aspects 1-4, comprising simultaneously transmitting data comprising (A) the real-time depth video stream of the ROI and the adjusted (B) real-time plot of motion for the ROI to one or more devices for radiation treatment.

6. The method of aspect 1, wherein the motion/position capture device comprises a mobile device or a smartphone with a depth sensor.

7. The method of any one of aspects 2-6, wherein the motion/position capture device comprises a mobile device or a smartphone with a depth sensor.

8. The method of aspect 6 or 7, wherein the smartphone operates on an operating system that is configured for sensing depth.

9. The method of aspect 8, wherein the operating system comprises an Apple™ operating system.

10. The method of aspect 8, wherein the operating system comprises an Android operating system.

11. The method of any one of aspects 1-10, wherein the device comprises a front-facing camera located on the same side of the device as the screen of the device.

12. The method of any one of aspects 1-10, wherein the device comprises a back-facing camera located on the backside of the device relative to the screen of the device.

13. The method of any one of aspects 1-10, wherein the device comprises a front-facing camera located on the same side of the device as the screen of the device and a back-facing camera located on the backside of the device relative to the screen of the device.

13. The method of any one of aspects 1-13, wherein the subject has a tumor located in the in the torso, neck or head.

14. The method of aspect 13, wherein the tumor is breast tumor, lung cancer, stomach cancer, intestinal cancer, colon cancer, or ovarian cancer.

15. The method of any one of aspects 1-14, wherein the device is connected to a data storage system, a remote display device, a radiation device, and/or a medical imaging device.

16. The method of aspect 15, wherein the data storage system comprises cloud storage, the remote display device comprises a computer monitor, laptop, smartphone, or another handheld device comprising a screen.

17. The method of aspect 15, wherein the data storage system comprises a medical device.

18. The method of aspect 17, wherein the medical device comprises a radiation therapy device for treatment with breathing maneuvers.

19. The method of aspect 18, wherein the radiation therapy device for treatment with breathing maneuvers comprises DIBH radiation treatment device.

20. The method of aspect 17, wherein the medical device comprises a computer-assisted tomography (CAT) scanner, a magnetic resonance imager, or positron emission tomography (PET) scanner.

21. The method of any one of aspects 1-20, further comprising prompting the subject to perform breathing maneuvers comprising holding breath prior to start of the capturing and/or after the start of the focusing.

22. The method of aspect 21, further comprising indicating visually or audibly to the subject a first period of time the subject performed breath hold based on the analysis of the (B) real-time plot of motion of the surface.

23. The method of aspect 22, comprising further prompting the subject to perform breath hold and indicating visually or audibly to the subject a second period of time the subject performed breath hold.

24. The method of any one of aspects 22-23, wherein the steps of prompting are repeated till the subject performs breath hold for a period of time that is determined clinically acceptable.

25. The method of aspect 24, wherein the period of time that is determined clinically acceptable is at least 20 seconds.

26. The method of aspect 21, further comprising indicating visually or audibly to a healthcare provider the periods of time the subject performed breath hold based on the analysis of the (B) real-time plot of motion of the surface.

27. The method of aspect 26, wherein the healthcare provider is present at a location remote to the subject.

28. The method of any one of aspects 26-27, wherein the method comprising uploading the (B) real-time plot of motion of the surface and/or the periods of time the subject performed breath hold to a data storage, wherein the data storage is accessible by the healthcare provider.

29. The method of any one of aspects 1-28, wherein the method comprises relaying exhalation after end of a breath hold by the subject to a radiation device and/or an imaging device.

30. The method of any one of aspects 1-29, wherein the method comprises instructing a radiation device to pause radiation being delivered to the ROI when the subject exhales after the end of breath hold.

31. The method of any one of aspects 1-18, wherein the method comprises instructing an imaging device to pause imaging of the region of interest when the subject exhales after the end of breath hold.

32. A non-transitory computer-readable medium comprising instructions stored thereon for causing a computer system to implement the methods of any one of Aspects 1 to 31.

33. A computer system comprising the non-transitory computer-readable medium of Aspect 32.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. All such modifications are intended to be included within the scope of the appended claims.

### Example 1

### An iOS Surface Audiovisual Biofeedback Smartphone Application for Respiratory Monitoring in Radiation Oncology

### Introduction

Paramount to the implementation of gating or breath-hold motion management in radiotherapy is the detection of respiratory signals using either internal or external probes to track and monitor respiratory motion (Bertholet J, et al., Physics in Medicine & Biology. 2019 Aug. 7; 64(15):15TR01). Furthermore, to improve the reproducibility of these methods, audiovisual feedback systems have been previously developed and shown to improve lung tumor position reproducibility and volume consistency (Park Y K, et al., Medical physics. 2011 June; 38(6 Part1):3114-24). Unfortunately, most clinical systems are sophisticated and complex, which could impede the widespread use of these systems especially in locations where staff and resources are limited. The iOS application proposed here is a simple-to-use, easy-to-implement low-cost alternative to commercially available products that monitor patient motion. This iOS application has the potential to facilitate the translation of respiratory gated techniques to centers that currently do not have access to respiratory motion management systems, such as lower-middle income countries (LMICs).

### Methods

The iOS application, coined iOS Surface Audiovisual Biofeedback (iSAVB), was developed in Swift (Apple Inc.) and implemented on an iPhone® X that has the TrueDepth camera capabilities.

The accuracy of motion traces was validated using the QUASAR™ Respiratory Motion Phantom (Modus Medical Devices). Motion was measured using iSAVB from previously recorded motion traces during free-breathing and breath-hold treatments which were programmed into the motion platform.

ISAVB measurement of displacement was compared to the input signal trace using linear regressions and Bland-Altman analysis.

### Results

An audiovisual feedback system that leverages depth information from the depth sensor onboard iOS devices was developed and its feasibility was assessed. The overall objective was to provide audiovisual coaching for improved patient compliance and radiation treatment.

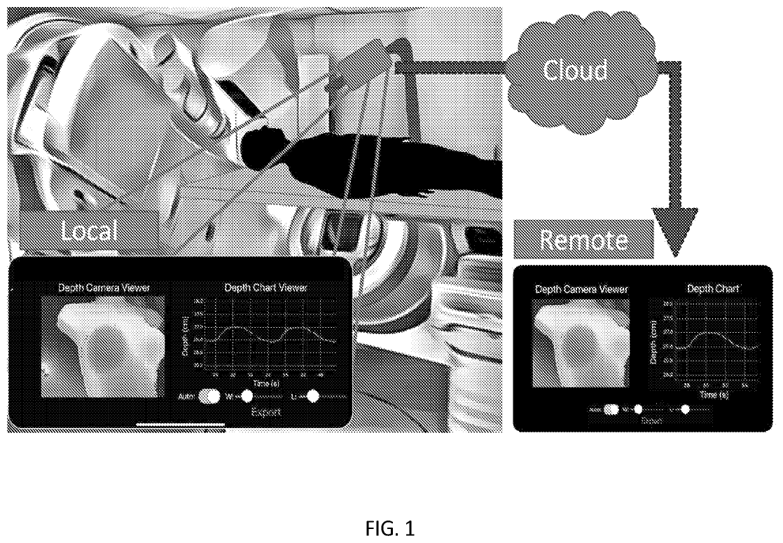

Development of iOS Application: The interface of the application, as shown in FIG. 1, has three main functions: 1) the depth camera viewer shows the camera feed with a depth color-map overlaid, 2) patient specific respiratory trace, and 3) save/record. This GUI can be used as an audio-visual feedback system and has the ability to window (W) and level (L) the viewer.

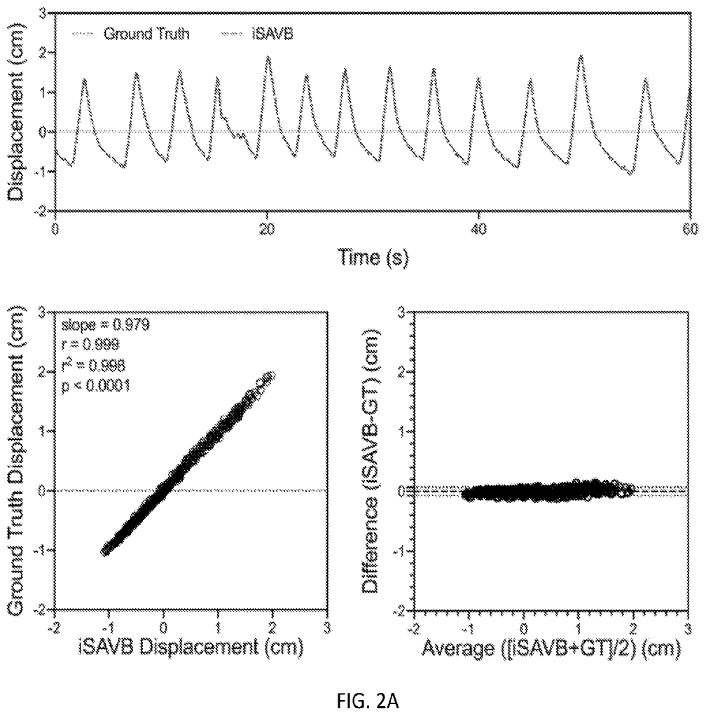

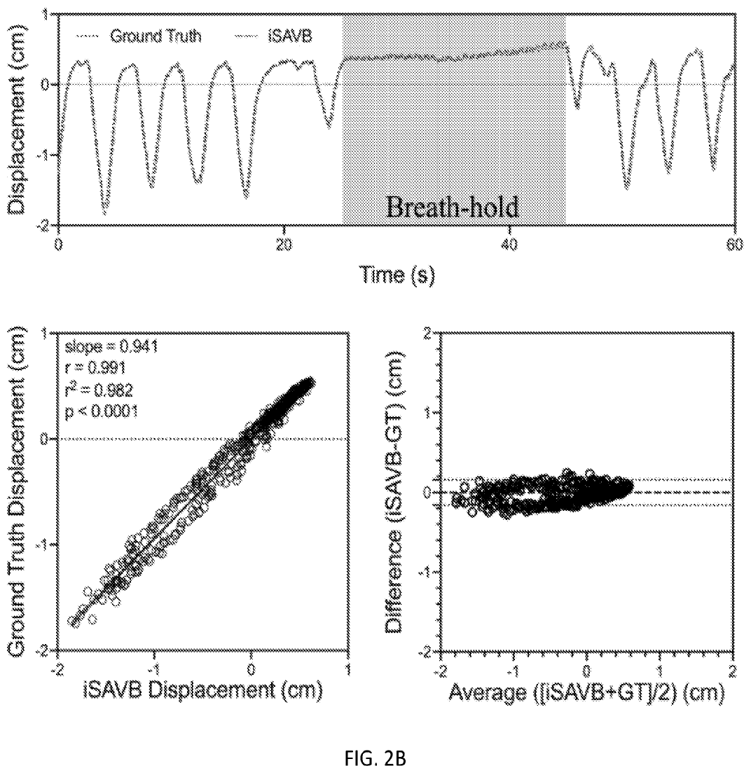

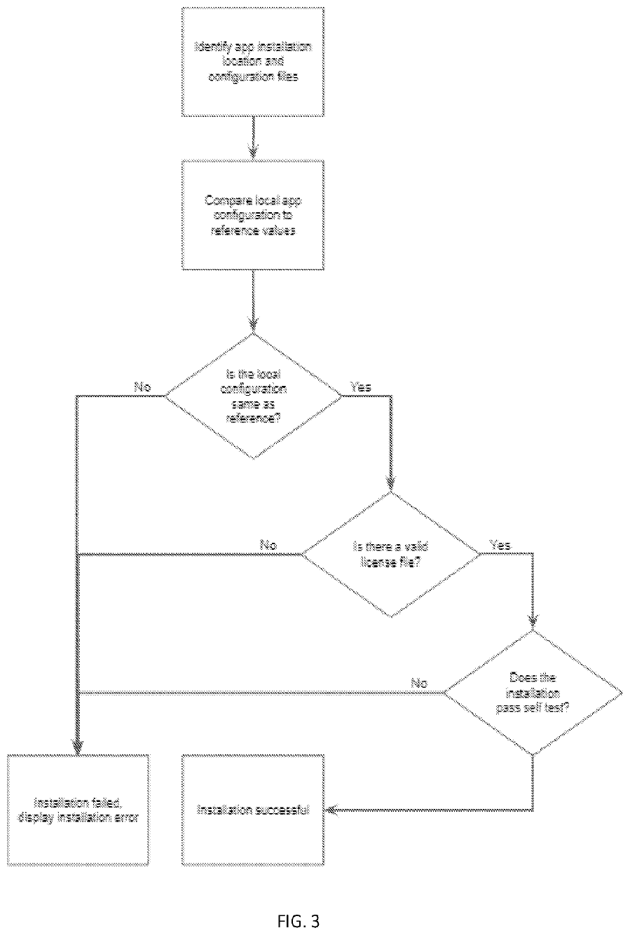

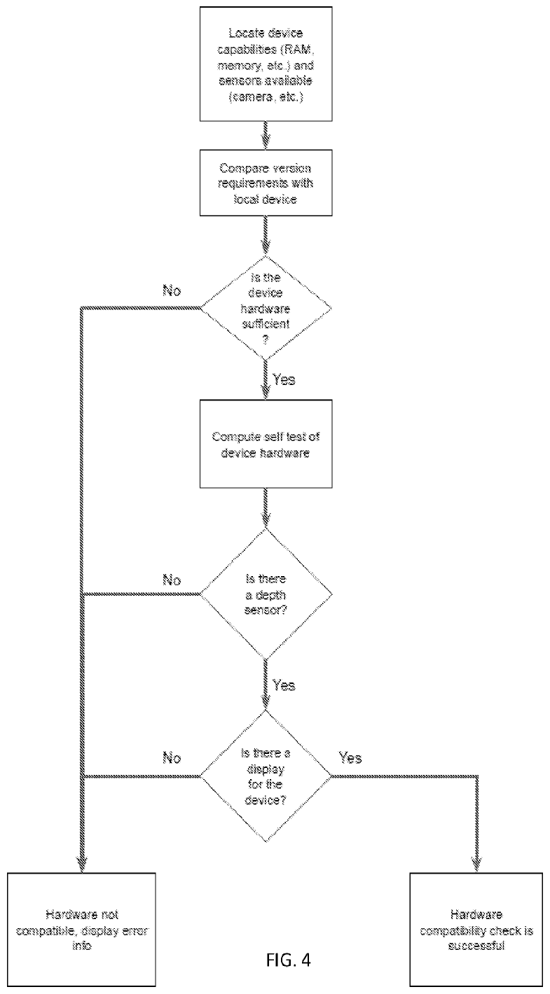

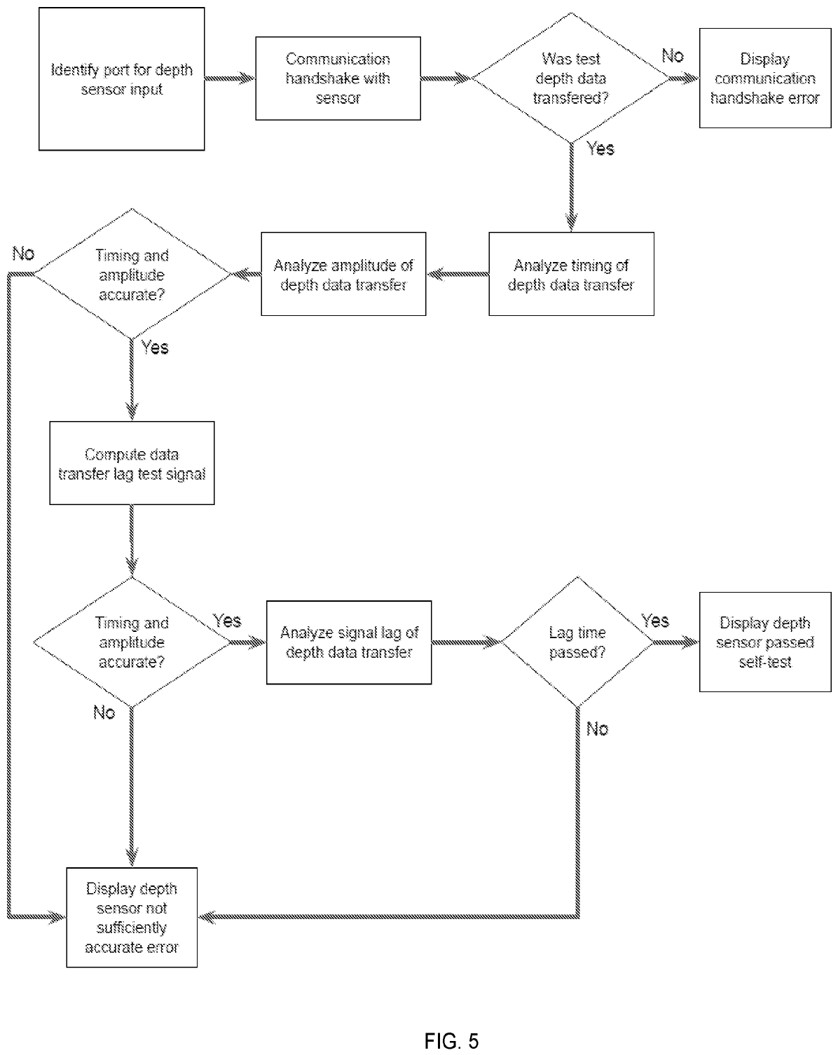

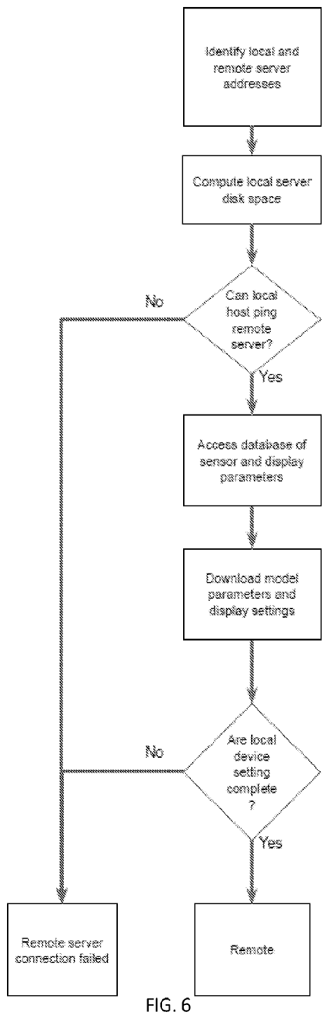

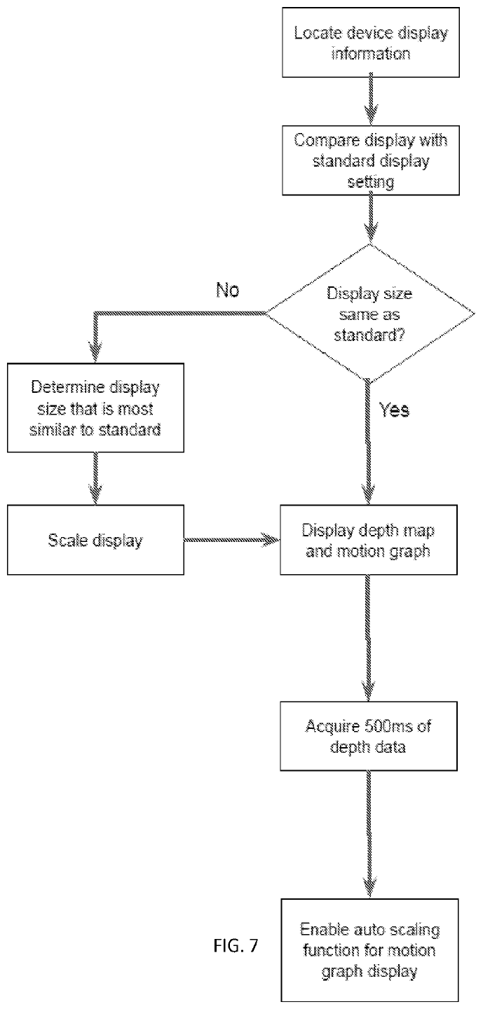

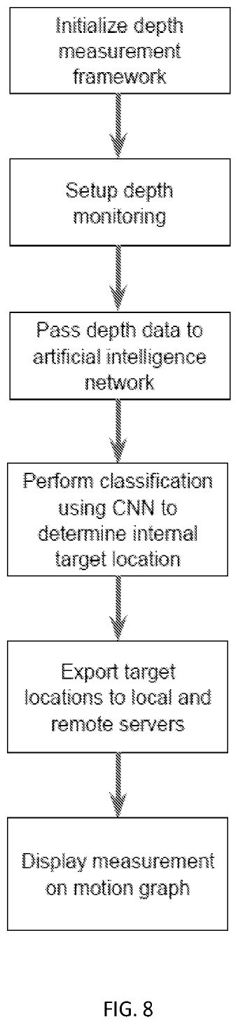

Evaluation of iOS Application using Programmable Motion Phantom: FIG. 2A and FIG. 2B show a comparison between two different patient traces that have been previously recorded with regular breathing and breath-hold. The patient traces were programmed in a dynamic phantom (QUASAR™, labelled as “Ground Truth”) and the iSAVB app was used to measure displacement as is plotted in the graph with dashed line. Excellent agreement is shown between iSAVB and programmed phantom motion (“Ground Truth”) with strong correlation and low bias in signal giving confidence that iSAVB is reliably measuring motion.

Feasibility of an iOS application to provide depth information for real-time respiratory motion monitoring was demonstrated using iOS depth camera signal processing. With the ubiquity of smartphone devices, this work can provide audiovisual biofeedback to patients in a low-cost platform or where integration of iOS devices will improve efficacy in radiation therapy.

What is claimed is:

1. A computer-implemented method comprising:
displaying on a screen of a motion/position capture device and/or on a screen remotely connected to the device:
(A) real-time depth video stream of the surface of the torso of a subject in a field of view of a camera of the motion/position capture device, and
(B) real-time plot of motion of the surface;
focusing the field of view to capture motion over a region of interest (ROI) on the surface;
capturing motion in the ROI over a period of time; and
displaying (A) the real-time depth video stream of the ROI and an adjusted (B) real-time plot of motion for the ROI, wherein the (B) real-time plot of motion for the ROI displays motion of chest and/or abdomen of the subject associated with inhalation and exhalation.

2. The method of claim 1, wherein capturing motion in the ROI over a period of time comprises simultaneously updating (B) real-time plot of motion for the ROI by applying an artificial intelligence (AI) to update motion metrics.

3. The method of claim 1 or 2, comprising simultaneously transmitting data comprising (A) the real-time depth video stream of the ROI and the adjusted (B) real-time plot of motion for the ROI to a remote server.

4. The method of any one of claims 1-3, wherein the screen remotely connected to the device is the screen of a remote monitoring device, a data storage device, a radiation treatment device, and/or an imaging device.

5. The method of any one of claims 1-4, comprising simultaneously transmitting data comprising (A) the real-time depth video stream of the ROI and the adjusted (B) real-time plot of motion for the ROI to one or more devices for radiation treatment.

6. The method of claim 1, wherein the motion/position capture device comprises a mobile device or a smartphone with a depth sensor.

7. The method of any one of claims 2-6, wherein the motion/position capture device comprises a mobile device or a smartphone with a depth sensor.

8. The method of claim 6 or 7, wherein the smartphone operates on an operating system that is configured for sensing depth.

9. The method of claim 8, wherein the operating system comprises an Apple™ operating system.

10. The method of claim 8, wherein the operating system comprises an Android operating system.

11. The method of any one of claims 1-10, wherein the device comprises a front-facing camera located on the same side of the device as the screen of the device.

12. The method of any one of claims 1-10, wherein the device comprises a back-facing camera located on the backside of the device relative to the screen of the device.

13. The method of any one of claims 1-10, wherein the device comprises a front-facing camera located on the same side of the device as the screen of the device and a back-facing camera located on the backside of the device relative to the screen of the device.

14. The method of any one of claims 1-13, wherein the subject has a tumor located in the in the torso, neck or head.

15. The method of claim 14, wherein the tumor is breast tumor, lung cancer, stomach cancer, intestinal cancer, colon cancer, or ovarian cancer.

16. The method of any one of claims 1-15, wherein the device is connected to a data storage system, a remote display device, a radiation device, and/or a medical imaging device.

17. The method of claim 16, wherein the data storage system comprises cloud storage, the remote display device comprises a computer monitor, laptop, smartphone, or another handheld device comprising a screen.

18. The method of claim 16, wherein the data storage system comprises a medical device.

19. The method of claim 18, wherein the medical device comprises a radiation therapy device for treatment with breathing maneuvers.

20. The method of claim 19, wherein the radiation therapy device for treatment with breathing maneuvers comprises DIBH radiation treatment device.

21. The method of claim 18, wherein the medical device comprises a computer-assisted tomography (CAT) scanner, a magnetic resonance imager, or positron emission tomography (PET) scanner.

22. The method of any one of claims 1-21, further comprising prompting the subject to perform breathing maneuvers comprising holding breath prior to start of the capturing and/or after the start of the focusing.

23. The method of claim 22, further comprising indicating visually or audibly to the subject a first period of time the subject performed breath hold based on the analysis of the (B) real-time plot of motion of the surface.

24. The method of claim 23, comprising further prompting the subject to perform breath hold and indicating visually or audibly to the subject a second period of time the subject performed breath hold.

25. The method of any one of claims 22-24, wherein the steps of prompting are repeated till the subject performs breath hold for a period of time that is determined clinically acceptable.

26. The method of claim 25, wherein the period of time that is determined clinically acceptable is at least 20 seconds.

27. The method of claim 22, further comprising indicating visually or audibly to a healthcare provider the periods of time the subject performed breath hold based on the analysis of the (B) real-time plot of motion of the surface.

28. The method of claim 27, wherein the healthcare provider is present at a location remote to the subject.

29. The method of any one of claims 26-28, wherein the method comprising uploading the (B) real-time plot of motion of the surface and/or the periods of time the subject performed breath hold to a data storage, wherein the data storage is accessible by the healthcare provider.

30. The method of any one of claims 1-29, wherein the method comprises relaying exhalation after end of a breath hold by the subject to a radiation device and/or an imaging device.

31. The method of any one of claims 1-30, wherein the method comprises instructing a radiation device to pause radiation being delivered to the ROI when the subject exhales after the end of breath hold.

32. The method of any one of claims 1-19, wherein the method comprises instructing an imaging device to pause imaging of the region of interest when the subject exhales after the end of breath hold.

33. A non-transitory computer-readable medium comprising instructions stored thereon for causing a computer system to implement the methods of any one of claims 1 to 3132

34. A computer system comprising the non-transitory computer-readable medium of claim 33.
